(19) United States
(12) Patent Application Publication
(10) Pub. No.: US 2009/0017197 A1
(43) Pub. Date: Jan. 15, 2009

(54) IrOx nanowire protein sensor

(75) Inventors: Fengyan Zhang, Camas, WA (US); Ravi K. Reddy, Portland, OR (US); Bruce D. Ulrich, Beaverton, OR (US); Shalini Prasad, Portland, OR (US); Sheng Teng Hsu, Camas, WA (US)

Correspondence Address: SHARP LABORATORIES OF AMERICA, INC.;C/O LAW OFFICE OF GERALD MALISZEWSKI, P.O. BOX 270829, SAN DIEGO, CA, 92198-2829, US

(73) Assignee: Sharp Laboratories of America, Inc.

(21) Appl. No.: 11/827,469

(22) Filed: Jul. 12, 2007

(51) Int. Cl. B05D 5/12; C12M 1/00

(52) U.S. Cl. 427/126.5; 204/403.01

(57) ABSTRACT

An iridium oxide (IrOx) nanowire protein sensor and associated fabrication method are presented. The method provides a substrate and forms overlying working and counter electrodes. A dielectric layer is deposited over the working and counter electrodes and contact holes are formed in the dielectric layer, exposing regions of the working and counter electrodes. IrOx nanowires (where 0≦X≦2) are grown from exposed regions of the working electrode. In one aspect, the IrOx nanowires are additionally grown on the dielectric, and subsequently etched from the dielectric. In another aspect, IrOx nanowires are grown from exposed regions of the counter electrode.

### BACKGROUND OF THE INVENTION

1. Field of the Invention

This invention generally relates to integrated circuit (IC) fabrication and, more particularly, to a fine resolution protein sensor fabricated with iridium oxide nanowire electrodes.

2. Description of the Related Art

The current industry standard for protein detection is fluorescent-based detection. Other detection means include: (1) Amperometry, (2) Potentiometry, and (3) Conductance. Table 1 highlights the advantages and shortcomings of these techniques for protein sensing.

TABLE 1
Comparison of Protein Sensing Technologies

| | Fluorescence | Potentiometry and |
|---|---|---|
| Capacitance Method | method | Amperometry |
| High sensitivity of | Low Sensitivity of | Low Sensitivity of |
| detection: | detection: | detection: |
| femto-molar range | nano to pico molar | nano to pico molar |
| Rapid response time: | Response time very | Response time high: |
| seconds to minutes | high: Hours to days | tens of minutes to |
| | | hours |
| High Signal-to-Noise | Low Signal to Noise | Low Signal to Noise |
| Ratio: Above 95% | Ratio: About 80% | Ratio: About 85% |
| Low consumption of | Higher consumption | High consumption of |
| power: Tens of μW | of power: Hundreds of | power: few Watts |
| (micro watts) | Watts | |
| Highly portable: Size | Low portability. Size | Not highly portable: |
| in millimeter to | in tens of centimeters | Size in centimeter scale |
| centimeter scale | to meters | |

From the above comparison, it can be seen that there are advantages to choosing the capacitance method of detection. At the nano-scale, the amount of surface area available for adherence is very high and the distance of separation between electrodes is reduced. Both these factors suggest to possibility of improved sensitivity, as capacitance is directly proportional to the surface area. To induce a change in capacitance, no additional reactions, such as reduction-oxidation reactions, need be instigated. Among electrical parameters, capacitance is the least affected by inherent background currents, making it a highly stable parameter for biomolecule detection.

Proteomics research has resulted in the identification of a large range of biomarkers that have the potential of greatly improving disease diagnosis. The availability of multiple protein markers is believed to be especially important in the diagnosis of complex diseases ranging from cancer identification to cardiovascular diseases. For these complex diseases, the heterogeneity of the disease makes tests of single protein markers inadequate. Patterns of multiple protein markers might, however, provide the information necessary for the robust diagnosis of disease in any person within a population. Moreover, the detection of markers associated with different stages of disease pathogenesis could facilitate early detection.

Widespread use of protein markers in healthcare will depend upon the development of techniques that will enable the rapid and selective detection of multiple markers. This goal has not yet been achieved by any of the existing detection methods that include the use of micro cantilevers, surface plasmon resonance, enzyme linked immunosorbant assays (ELISA). and carbon nanotube based sensors.

Nanomaterials have been used to improve sensitivity in the nanogram sensitivity regime using silicon nanowire and carbon nanotube based devices. However, these materials are not suitable for multiplexed detection due to the complexity associated with the device fabrication and issues with repeatability.

FIG. 1 is a partial cross-sectional view comparing a conventional flat electrode with an electrode array (prior art). Micro-machined neural-stimulating electrode array technology has also been researched. The micro-machined electrode has the advantage of providing additional surface area to decrease the current density, while increasing the electrode density and avoiding material corrosion. However, a key issue to be resolved is the fabrication of an electrode array that can conform to concave shapes. For example, such as array would need to be formed on a flexible substrate (e.g., polyimide).

Another limitation associated with micromachining technology is size, as the individually machined electrodes cannot be made to a nano-size resolution. Even if a template of nano-sized structures could be micro-machined, plating an array of nanostructures, with a noble metal for example, in a sufficiently high aspect ratio is a big challenge. Micro-machined electrodes are normally formed from a thick film that is deposited using a physical vapor deposition (PVD) process or electrode plating. In either case, the resultant film, and micro-machined electrode post are typically a polycrystalline material.

IrOx nanowire-based electrodes have a better surface-to-volume ratio, as compared to carbon nanotubes (CNTs) for example, as well as a high resolution stimulation, biocompatibility, and ability to grow on transparent conducting electrodes such as ITO, SnO2, ZnO and TiO2 with or without any doping. Single-crystal IrO2 nanowires/rods/tips have a much longer life than polycrystalline IrO2, due to their higher chemical reaction resistance. Single-crystal IrOx nanostructures also have a higher conductance than polycrystalline IrO2, so they can pass through current more efficiently. However, it is difficult to form single-crystal IrO2 films using conventional PVD or electrode plating methods. IrO2 nanostructures can be formed using a solution method, but these structures have a low mechanical strength and poor crystal quality. Vapor phase transport methods can also be used to form IrO2 nanostructures, but this process requires high substrate temperature, and it is not suitable for use with glass and polyimide substrates.

It would be advantageous if a sensor could be fabricated using IrOx nanowire electrodes for capacitively measuring the detection of proteins.

### SUMMARY OF THE INVENTION

This disclosure presents integrated nanowire arrays in which distinct nanowire surfaces can be integrated with distinct receptors/antibodies, to function as individual components of device elements. The detection technique is such that the variation in the electrical conductivity associated with the binding of specific proteins onto selectively functionalized nanowire arrays can be measured. The result is an electrochemical signature that is unique to a specific antibody or protein/antigen pair. Non-specific bindings can be eliminated by such a technique, thus reducing background noise effects considerably and improving the signal to noise ratio. The individual device elements, due to their specific functionalization, are able to detect specific proteins. Hence, a large array of proteins can be detected in a matrix format within a few minutes i.e., in near real time as opposed to conventional detection methods that range from a few hours to a few days. The quantity of test sample required for such detection process is in the order of microliters, as compared to milliliters in the conventional methods.

Iridium oxide nanowires are used as the active elements in the detection process. These nanowires are biocompatible, and amenable to the addition of multifunctionality suitable for multiplexed detection. The large surface area afforded by these nanowires enables a reduction in the device footprint by increasing the active area for detection. Comparing to a planar IrO2 electrode, IrO2 nanowires have an improved surface-to-volume ratio, resulting in a high selectivity, high sensitivity larger linear dynamic range of detection, and rapid response time.

As noted above, iridium oxide has very good conductivity and charge storing capacity. As such, it can be used to detect even a very small change in surface charge. High selectivity can be achieved by incorporating protein receptors (antibodies) on the nanowires, which bind only to specific proteins. This binding induces a change in surface charge, on the nanowire surface. This change in surface charge is due to the modification of the surface charge of the proteins as a result of the binding, which can be efficiently detected. This technique is extremely sensitive to these surface charge variations, enabling the detection of very small concentrations of proteins.

Accordingly, a method is provided for forming an iridium oxide (IrOx) nanowire protein sensor. The method provides a substrate and forms overlying working and counter electrodes. A dielectric layer is deposited over the working and counter electrodes, and contact holes are formed in the dielectric layer, exposing regions of the working and counter electrodes. IrOx nanowires (where 0<X≦2) are grown from exposed regions of the working electrode. In one aspect, the IrOx nanowires are additionally grown on the dielectric, and subsequently etched from the dielectric. In another aspect, IrOx nanowires are grown from exposed regions of the counter electrode.

The working and counter electrodes may be a material such as ITO, SnO2, ZnO, TiO2, doped ITO, doped SnO2, doped ZnO, doped TiO2, TiN, TaN, Au, Pt, or Ir. The substrate may be Si, SiO2, quartz, glass, or polyimide. The dielectric layer may be made from a material such as SiO2 or SiN.

The contact holes have openings about equal to, and aligned with top surfaces of the underlying working and counter electrodes. The counter electrode has a top surface area in a range of 1 square micron to 1 square millimeter (mm2), and the working electrode has a top surface area in a range of about 1 to 1000 times smaller than the counter electrode top surface area. Both electrodes come in a variety of shapes, and are typically separated by a distance in a range between 0.1 and 10 microns.

Additional details of the above-described method, a method for capacitively detecting the presence of proteins, and an IrOx nanowire protein sensor array are presented below.

### BRIEF DESCRIPTION OF THE DRAWINGS

FIG. 1 is a partial cross-sectional view comparing a conventional flat electrode with an electrode array (prior art).

FIGS. 2 and 3 are partial cross-sectional and plan views, respectively, of an iridium oxide (IrOx) nanowire protein sensor array.

FIG. 4 is a partial cross-sectional view of the sensor of FIG. 2.

FIG. 5 is a plan view depicting various electrode shapes and orientations.

FIG. 6 is a plan view of the entire sensor, showing an array electrode pairs.

FIG. 7 is a diagram representing a binding process where each antigen is uniquely shaped to fit a corresponding antibody.

FIG. 8 is a diagram depicting the phenomenon of an electrical double layer.

FIG. 9 is a perspective view of an exemplary electrode design.

FIGS. 10A through 10E depict steps in the fabrication of a protein sensor array.

FIG. 11 is a scanning electron microscope (SEM) optical image of a working and counter electrode pair showing the growth of IrOx nanowires.

FIG. 12 is a plan view of an exemplary arrangement of electrode pairs in a protein sensor array.

FIGS. 13A through 13D depict steps in the fabrication of a protein sensor with IrOx nanowires grown on the working electrode, but not on the counter electrode.

FIG. 14 is a diagram depicting the use of linkers to bind a protein to an IrOx nanowire.

FIG. 15 is a diagram depicting a relationship between the binding process and capacitance measurement.

FIG. 16 is a flowchart illustrating a method for forming an iridium oxide (IrOx) nanowire protein sensor.

FIG. 17 is a flowchart illustrating a method for using capacitance measurements to detect the presence of proteins in an ambient environment.

FIG. 18 is a diagram depicting antibody saturation measurements with, and without blockers. A slight increase in impedance with the addition of blockers denotes the binding of BSA with unused sites, which reduces non-specificity.

FIG. 19 is a diagram depicting dose response measurements with, and without blockers.

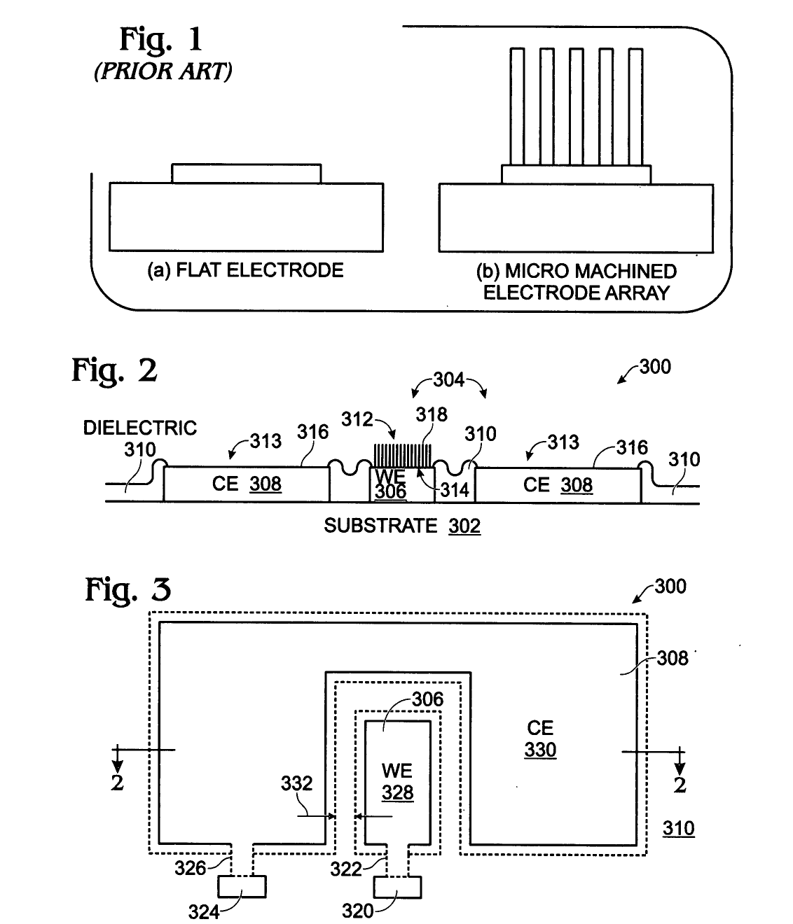

### DETAILED DESCRIPTION

FIGS. 2 and 3 are partial cross-sectional and plan views, respectively of an iridium oxide (IrOx) nanowire protein sensor array. The sensor array 300 comprises a substrate 302 and a plurality of electrode pairs 304. For simplicity, FIGS. 2 and 3 show a single pair. Each electrode pair, as represented by pair 304, includes a working electrode (WE) 306 overlying the substrate 302 and a counter electrode (CE) 308 overlying the substrate 302. A dielectric layer 310 overlies the working and counter electrodes 306/308. Contact holes 312 and 313 are formed in the dielectric layer 310, exposing regions 314 and 316, respectively, of the working electrode 306 and the counter electrode 308. IrOx (0<X≦2) nanowires 318 are grown from exposed region 314 of the working electrode 306. In some aspects, single-crystal IrO₂ nanowires are grown. The electrode pair 304 may further include a coating of a material such as antibody linker molecules, antibodies, protein blocker agents, or combinations of the above-mentioned materials (not shown in this figure). Note: in some aspects, the coating is applied prior to testing, as opposed to at the time of sensor fabrication.

A nanowire may alternately be known as a nanostructure, nanorod, nanotip, or nanotube. The average IrOx nanowire has an aspect ratio in a range of about 1:1 to about 1000:1. As used herein, aspect ratio is defined as the ratio of the nanowire height, to the nanowire diameter or width at the base, where it is attached to the electrode. The IrOx nanowires have an average height in the range of about 10 nanometers (nm) to about 10 micrometers (am). The IrOx nanowires have an average base end diameter in the range of about 1 nm to about 1 μm.

The working and counter electrodes 306/308 are a material such as ITO, SnO₂, ZnO, TiO₂, doped ITO, doped SnO₂, doped ZnO, doped TiO₂, TiN, TaN, Au, Pt, or Ir. The substrate 302 may be a material such as Si, SiO₂, quartz, glass, or polyimide. The dielectric layer 310 may be a material such as SiO₂ or SiN. However, it should be noted that the list of above-mentioned materials are examples of materials that are already conventionally used in many IC fabrication processes, and that the sensor device 300 may be enabled with other materials that would be well known in the art.

As seen in FIG. 3, the substrate is a substrate chip with edges (only one edge can be seen in this view). Electrode pair 304 further includes probe pads along the chip edges, and traces connecting the electrodes to the probe pads. Probe pad 320 is connected to the working electrode 306 via trace 322. The trace and electrode edges are shown in phantom, as they are covered by dielectric 310. Probe pad 324 is connected to counter electrode 308 via trace 326. The contact holes 312 and 313 have openings about equal to, and aligned with top surfaces 328 and 330, respectively, of the underlying working and counter electrodes 306/308.

The counter electrode 308 has a top surface 330 area in a range of 1 square micron to 1 square millimeter (mm²). The working electrode 306 has a top surface 328 area in a range of about 1 to 1000 times smaller than the counter electrode top surface area. Both the working and counter electrodes 306/308 come in a variety of shapes and orientations, depending upon the specific function of the sensor. As shown, both electrodes 306/308 have a substantially rectangular shape, and the counter electrode substantially surrounds the working electrode. In other aspects not shown, the traces are carried in layers either overlying or underlying the electrodes, and connected to the electrodes using vias. Typically, the working electrode 306 is separated from its corresponding counter electrode 308 by a (minimum) distance 332 in the range between 0.1 and 10 microns. However, the distance 332 need not necessarily be uniform.

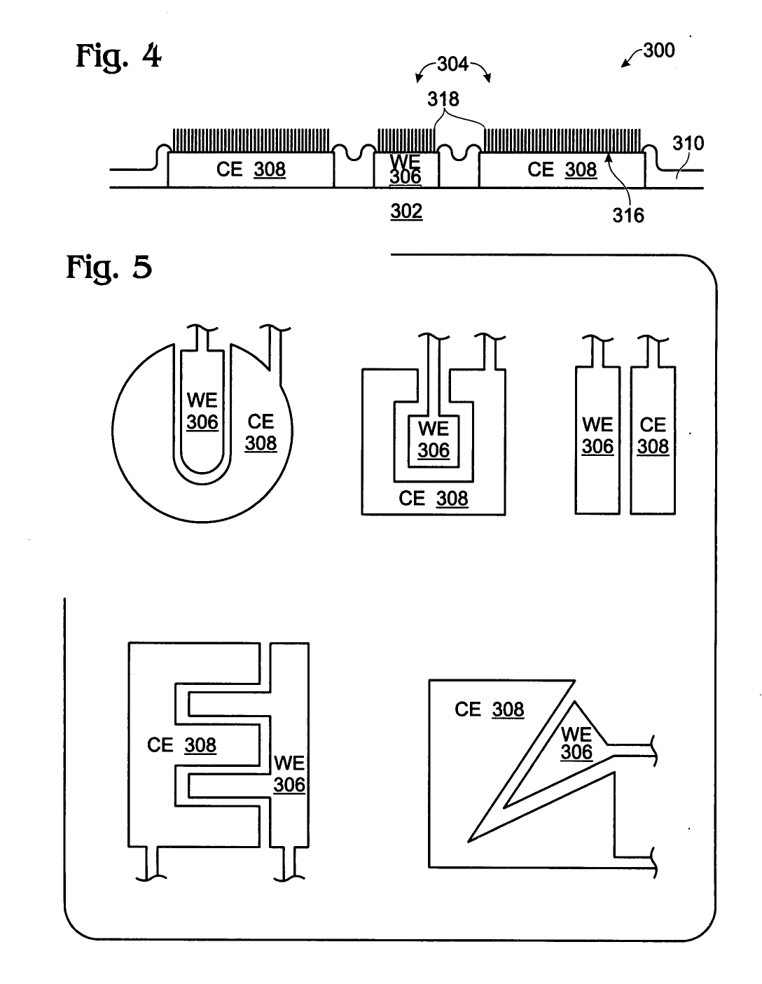

FIG. 4 is a partial cross-sectional view of the sensor of FIG. 2. In this aspect, IrOx nanowires 318 are also grown from the exposed region 316 of the counter electrode 308.

FIG. 5 is a plan view depicting various electrode shapes and orientations. As shown, both the working electrode 306 and the counter electrode 308 may have a variety of shapes, such as circular, rectangular, hexagonal, and oval. However, other shapes are also feasible. Further, the working electrode 306 need not necessarily have the same shape as the counter electrode 308. The working electrode 306 and the counter electrode 308 are arranged in an orientation such as adjacent, the counter electrode substantially surrounding the working electrode, and with an interdigital separation pattern.

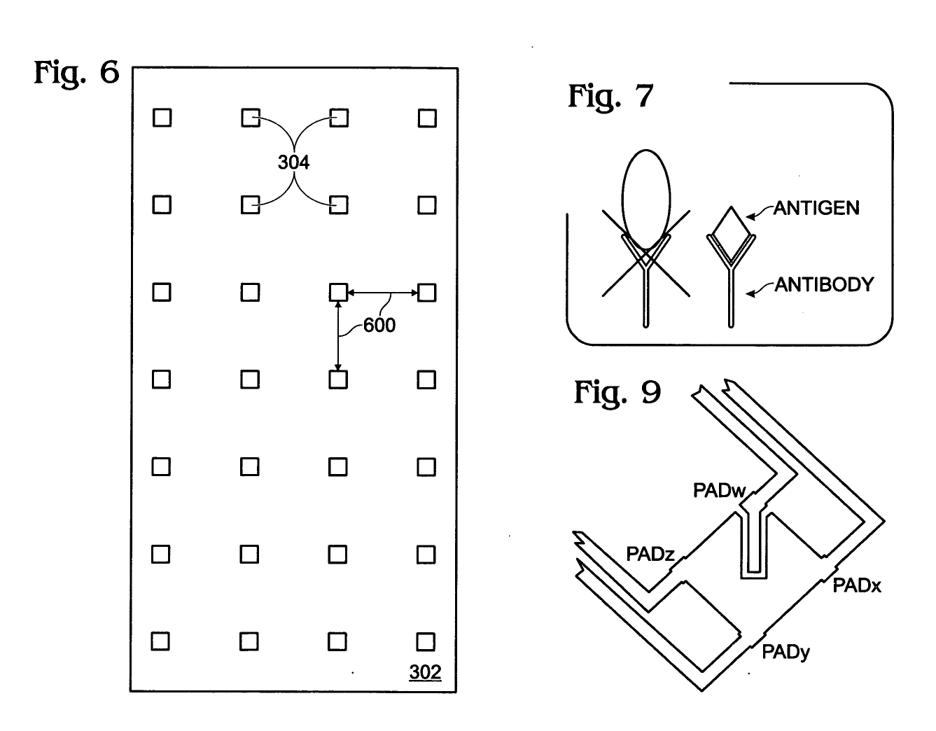

FIG. 6 is a plan view of the entire sensor, showing an array electrode pairs. The substrate chip 302 has a surface area in the range of 1 mm² to 1000 mm². Typically, the array includes between 2 and 128 electrode pairs 304. Each pair 304 is separated by a (minimum) distance 600 in the range of 1 to 500 microns. The pairs may be arranged in a grid pattern, as shown, or in a circular or concentric ring pattern (not shown). The array is not necessarily limited to any particular number of electrode pairs, separation of pair, or pattern of pairs. The distance between electrode pairs on the substrate may be uniform or varied, and may be measured from the substrate center or from adjacent clusters. For example, the distance between electrode pairs may be a function of the relative position of the pair from the substrate center, in which case the array pattern is likely to be circular or concentric rings. Note: the probe pads and traces are not shown in this view.

### FUNCTIONAL DESCRIPTION

One limitation to the capacitance measuring technique is its dependence upon the protein receptor specificity. However, covalent linker chemistry can be incorporated to promote greater specificity. To characterize protein detection based on capacitance measurements, the following measurement technique may be employed: (1) Baseline and Control Measurements, (2) Protein Receptor (Antibody) Saturation Measurements, and (3) Protein (Antigen) binding measurements.

FIG. 7 is a diagram representing a binding process where each antigen is uniquely shaped to fit a corresponding antibody. Protein detection is a two-part process. It involves the binding of the protein (antigen) to an immobilized protein receptor (antibody). Antibodies and antigens are proteins, which have lock-and-key like structures, wherein one antibody binds to one specific antigen. When the antibody binds with the antigen, the charge associated with the proteins changes and, hence, the change in charge is used as the signal of detection.

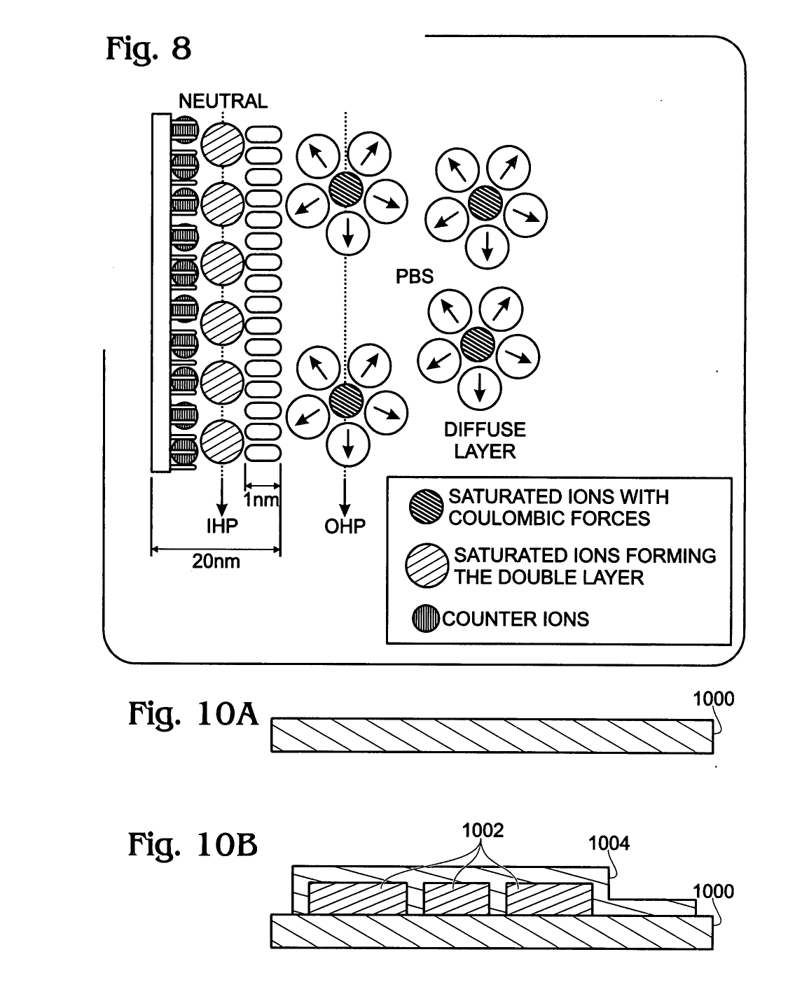

FIG. 8 is a diagram depicting the phenomenon of an electrical double layer. An electrical double layer is a phenomenon that occurs at a solid-liquid interface. Ions of one charge type are fixed to the surface of the solid, and an equal number of mobile ions of the opposite charge are distributed through the neighboring region of the liquid. The result is absolutely analogous to an electrical capacitor that has two plates of charge separated by some distance (d) with a potential drop occurring in a linear manner between the two plates.

### Gouy-Chapman Double Layer

The thickness of the diffuse double layer at room temperature is derived as,

λ_{double}=3.3×10⁶∈ᵣ/(zc^{1/2}) (1)

Where, ∈ᵣ is the relative permittivity of the medium
Z is the charge on the ion (valence)
C is the concentration of ions.

From this equation it can be seen that the double layer thickness decreases with increasing valence and concentration. It can also be seen that the properties of the electrical double layer depend on the surface charge on the electrode, the DC bias voltage applied, the concentration of ions in the solution, and the charge of the individual ions.

Capacitance is inversely proportional to the double layer thickness:

Cα1/λ_{double}

Hence the capacitance induced can be sufficiently enhanced by using a high concentration ionic solution. Typically, the thickness of the double layer can be changed by varying the bias voltage and sweeping it over a finite frequency range, to find the point at which there is high capacitance. During sensor operation, a potential is applied between the two electrodes and the electrical double-layer formed at each electrode results in a localized capacitance. These capacitors may be represented as working and counter electrodes. The surface area of counter electrode is typically many times the area of the working electrode, to maximize the protein binding signal on the working electrode.

FIG. 9 is a perspective view of an exemplary electrode design. Some of the parameters in the sensor device design include the distance of separation between electrodes, the surface area of each electrode, the size and shape of the working and counter electrodes, and dielectric in between electrodes. As shown, the smaller electrode acts as a working electrode, the larger one is referred to as the counter electrode. A bias may be applied to the counter electrode (or both electrodes) to create the potential across the electrode. Due to the size variation in the electrodes (in some aspects, the counter electrode is ˜10 times the size of the working electrode), the variation in the capacitance of the smaller electrode is larger than one the counter electrode. IrOx nanowires may be grown on the surfaces of working electrode and counter electrode. Alternately, IrOx nanowires are only grown from one of the electrodes.

The smaller the distance of separation between electrodes, the greater the effect of the capacitive measurements. In many circumstances, a distance of less than 2 um is desired. In one aspect, 10 sets of complimentary electrodes with IrOx nanowires, with sizes in the range of 1-50 microns for each electrode, provide enough surface area to detect proteins down to the picogram per milliliter (pg/ml) range.

In another aspect, the working electrode and counter electrode can be arranged in a multi-fingered interdigital pattern. The sizes of the working and counter electrodes can be the same, or different. The electrodes act as the plates of a capacitor, and the change in the charges from the proteins changes the capacitance measurement in a manner that is unique for each protein.

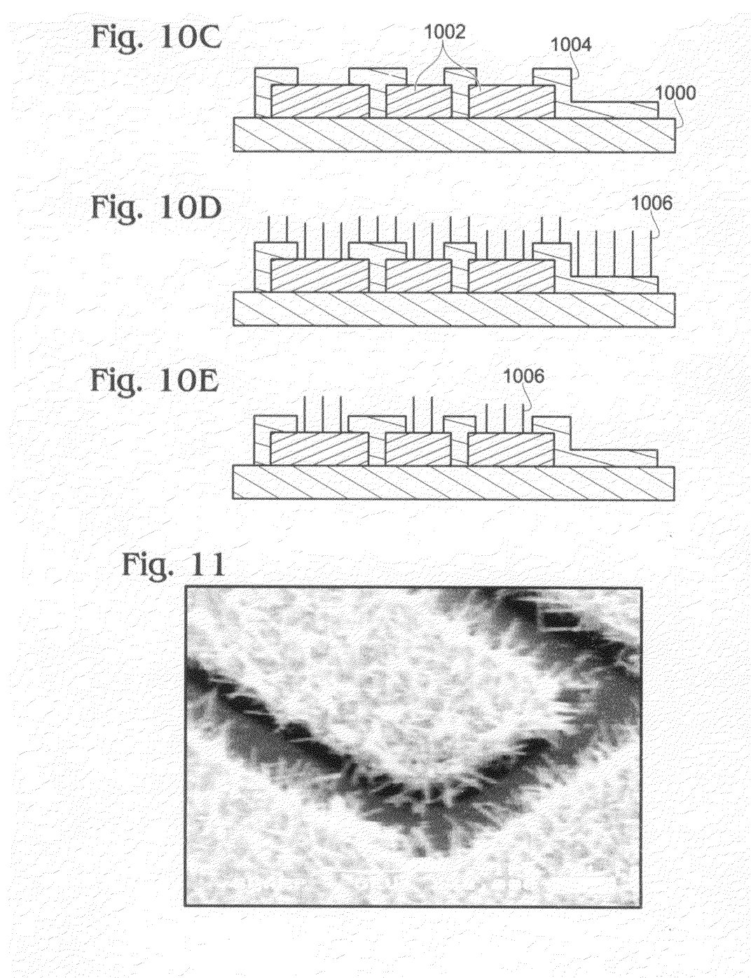

FIGS. 10A through 10E depict steps in the fabrication of a protein sensor array. First, the process begins with a Si, SiO₂, quartz, or plastic (e.g., polyimide) substrate 1000, see FIG. 10A. In FIG. 10B a conductive layer 1002 such as Pt, Au, Ir, TiN, TaN or transparent conductive oxide (TCO), such as ZnO, TiO₂, ITO, SnO₂, or a doped conductive oxide is deposited on the substrate. Then, the conductive layer is annealed and patterned using wet or dry etching process. If ITO is used, an optional annealing process can be performed in oxygen at 200-600° C. for 10 to 3600 seconds to improve the transparency of the ITO film. After patterning the conductive layer, a dielectric layer 1004 such as SiO₂ or SiN is deposited on the wafer to passivate the conductive lines. In case of ITO, wet etching may be performed using an HCl based solution.

In FIG. 10C, contact holes to the conductive lines are etched out using either a dry or wet etching process. In case of SiO₂ or undensified SiN, an HF based solution can be used to open up the contact holes. After etching, the wafer is transferred to an IrOx chamber to grow IrOx nanowires 1006, see FIG. 10D. In one aspect, the IrOx nanowires grow on the conductive electrode surface and also on the dielectric surface. A second wet etching process is used to stripe the field IrOx nanowires by etching the underlying dielectric, leaving the IrOx nanowires in the contact holes, see FIG. 10E. If SiO₂ or undensified SiN are used as the dielectric, an HF based solution also works to strip the field IrOx nanowires. At this step, only a partial layer of the dielectric layer is striped away, leaving enough of a thickness to passivate the conductive lines on the field.

FIG. 11 is a scanning electron microscope (SEM) optical image of a working and counter electrode pair showing the growth of IrOx nanowires.

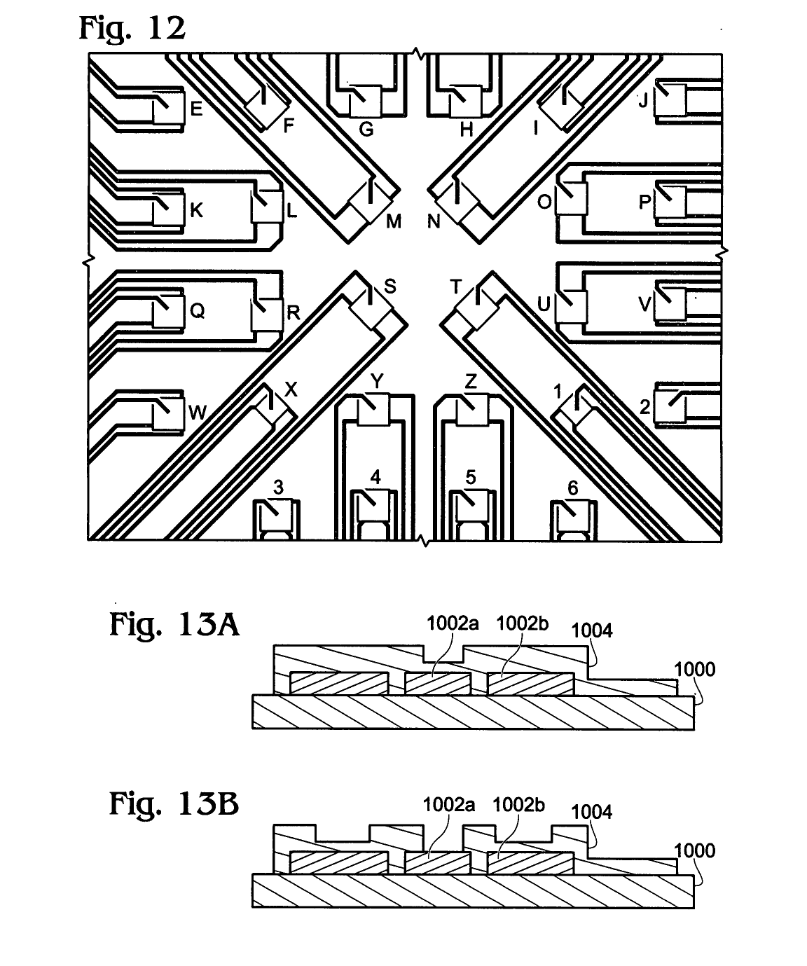

FIG. 12 is a plan view of an exemplary arrangement of electrode pairs in a protein sensor array.

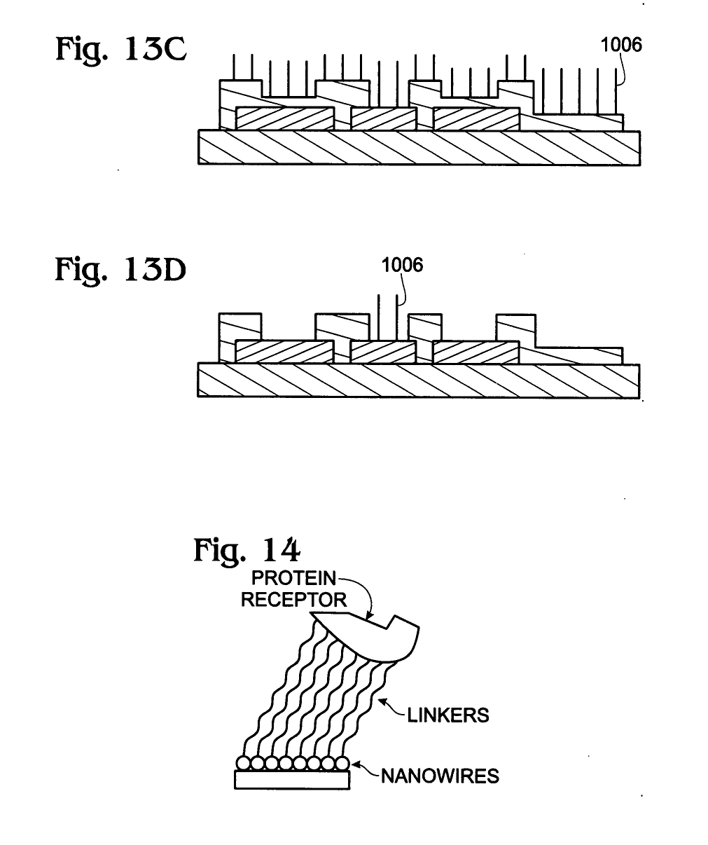

FIGS. 13A through 13D depict steps in the fabrication of a protein sensor with IrOx nanowires grown on the working electrode, but not on the counter electrode. In FIG. 13A, a contact hole is started on the working electrode 1002a surface only, etched about half way through the dielectric 1004 (e.g., 10 nm-100 nm). Then, contact holes on the working electrode 1002a, counter electrode 1002b, and probing pad (not shown) are etched together. A wet etching process can be used to expose just the conductive layer surface of the working electrode 1002a, but not at the counter electrode 1002b and the probing pads, see FIG. 13B. In FIG. 13C, IrOx nanowires 1006 are grown on the working electrode 1002a (on the surface of exposed conductive layer), on the dielectric 1004 covering the counter electrode 1002b and probing pad, and on the field dielectric 1004. Then, a wet etching process strips the IrOx nanowires 1006 on the dielectric 1004, including the IrOx nanowires overlying on the counter electrode 1002b and probing pad, since both are still covered with dielectric. The wet etching process is controlled to expose the conductive layer surface of the counter electrode 1002b and probing pads, see FIG. 13D.

### Protein Detection

Baseline Measurements: To find and offset the background capacitance, an impedance analyzer may be used. An impedance analyzer measures impedance as a function of frequency. Measurement probes are placed on the working electrode and the counter electrode, and connected to the impedance analyzer after basic calibration. The impedance measurements, cross-referenced to frequency, can be substituted in an equivalent circuit (mathematically) to find the capacitance. AC impedance and, hence, capacitive reactance is observed from the analyzer by varying the frequency sweep and bias voltages, which is then substituted in an equivalent circuit to obtain the effective capacitance.

At a particular frequency the capacitance reaches a local maximum due to the reduction of the double layer thickness. That capacitance value and its corresponding frequency are noted.

### Phosphate Buffered Saline (PBS) and DI Water

PBS is a buffer solution containing sodium chloride, sodium phosphate, and potassium phosphate. It is filled with ions, and the ionic strength of PBS increases with its concentration. To obtain the best capacitance, different concentrations of PBS (1X and 0.1X) are dropped onto the samples and the capacitive measurements are made at prescribed time intervals. De-ionized (DI) water, as the name signifies, is devoid of ions. So, the capacitance can also be measured when DI water acts as the medium to provide another reference marker.

### Antibody Immobilization

FIG. 14 is a diagram depicting the use of linkers to bind a protein to an IrOx nanowire. Linkers are used to make the antibodies adhere firmly to the iridium oxide nanowires. Alkanethiols are typical linker molecules and they bind well with iridium oxide, as they are self-assembled monolayers. In addition to alkanethiols, carboxylic acids, organosilicon derivatives, and diphosphonates act as good linkers on iridium oxide surfaces.

### Antibody Immobilization

Antibodies are immobilized on to the iridium oxide nanowires by placing them in an incubation chamber for about 30 minutes, at about 60° C. Once they are attached to the iridium oxide nanowires, the nanowires tend to change the equilibrium state of charge by using up some ions for binding. These changes in the surface charge on the nanowires change the overall capacitance measured between the electrodes.

Antibodies are added until the capacitance saturates and stabilizes. That capacitance measured provides a capacitance reference for the characterization for a particular protein.

### Antigen Binding

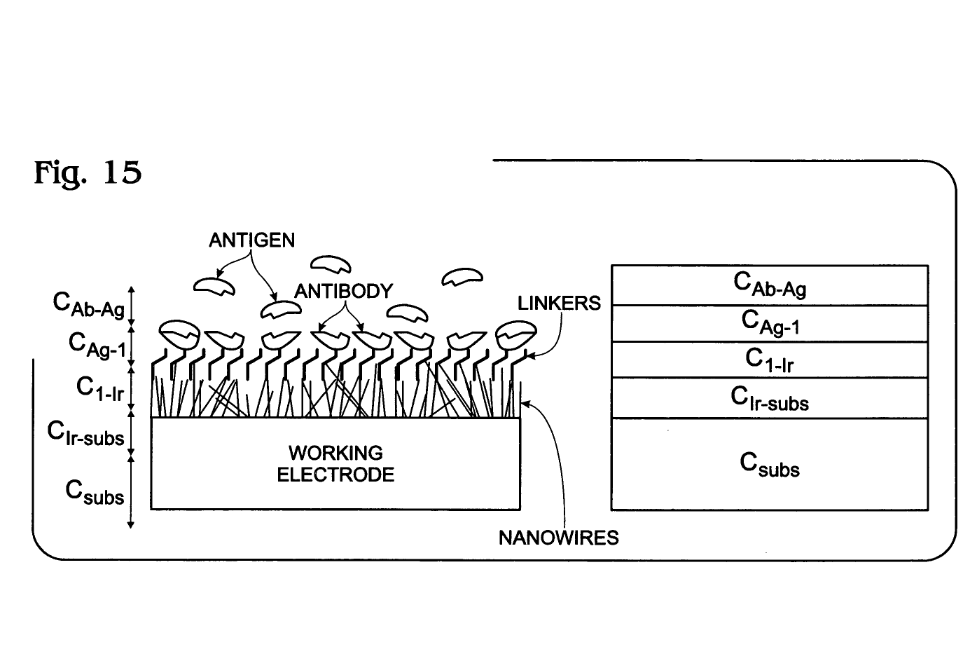

FIG. 15 is a diagram depicting a relationship between the binding process and capacitance measurement. All the capacitances are in series due to the capacitive interactions between different interfaces. C_{subs} is the substrate capacitance. C_{Ir-subs} is the capacitance between the nanowires and the substrate. C_{I-Ir} is the capacitance between the linkers and the nanowires. C_{Ab-1} represents the capacitance induced between the linkers and the antibodies. One capacitance of interest, C_{Ab-Ag}, is the capacitance induced between the antibody and the antigen when binding occurs.

Using baseline measurements, the substrate capacitance and C_{Ir-subs} are known. After adding linkers and adhering the antibodies on to the nanowires, all the capacitances, except the antigen-induced capacitance, can be found. Hence, the overall capacitance before binding is:

1/Cᵢₙᵢ=1/C_{subs}+1/C_{Ir-subs}+1/C_{l-Ir}+1/C_{Ab-l}

After exposing the nanowires to an environment that contains antigens, Ab—Ag binding takes place to induce the C_{Ab-Ag} capacitance to give:

1/C_{fin}=1/Cᵢₙᵢ+1/C_{Ab-Ag}

Hence, the difference between Cᵢₙᵢ and C_{fin} can be used to characterize protein detection. Known concentrations of proteins can be dropped onto the samples, and capacitive measurements taken for different concentrations, for the purpose of calibration.

### Blocking Analysis in Protein Detection

A blocker or a blocking agent is one which blocks all the unused sites on the nanowires to reduce the amount of nonspecific binding of proteins. When the working electrode is saturated with antibodies, most of the sites are used for binding. However, there may still be certain unoccupied sites that act as active binding sites for proteins when dropped on the electrodes. This condition brings about non-specific binding and increases background interference. Nonspecific binding is relevant due to the presence of other proteins in clinical samples (e.g., blood samples).

In one aspect, a blocking buffer, bovine serum albumin (BSA) is used to bind with these unreacted sites and improve the sensitivity of detection by reducing the background interference. BSA binds well with all potential sites of nonspecific interaction, without altering or obscuring the epitope for antibody binding.

FIG. 18 is a diagram depicting antibody saturation measurements with, and without blockers. A slight increase in impedance with the addition of blockers denotes the binding of BSA with unused sites, which reduces non-specificity.

FIG. 19 is a diagram depicting dose response measurements with, and without blockers. At lower concentrations, there is a constant increase in sensitivity and much higher sensitivity is achieved in the higher concentrations. With the addition of blockers, the sensitivity is found to increase by reducing the non-specific binding and background interference. Different concentrations of BSA may be analyzed to find the ideal concentration at which the blocking is most effective and the sensitivity is highest.

For example, some conventional limits of detection are ˜0.5 nanograms per ml (ng/ml), with a dynamic range of detection in the range from the lower ng/ml to higher micrograms per ml (μg/ml). Conventional frequency sweeps are in the range of ˜10 kHz to ˜3 MHz. The use of blockers as mentioned above, improves the lower limits of detection, which increases the dynamic range, as the blockers reduces background interference and non-specificity. Further, the sweep of frequency is extended to lower frequencies, as the double layer capacitance dominates in the lower frequency range. Again, this result yields higher values and higher sensitivity. As mentioned above, covalent linkers help in covalently binding the antibodies to the nanowires, hence, increasing stability and the dynamic range of detection.

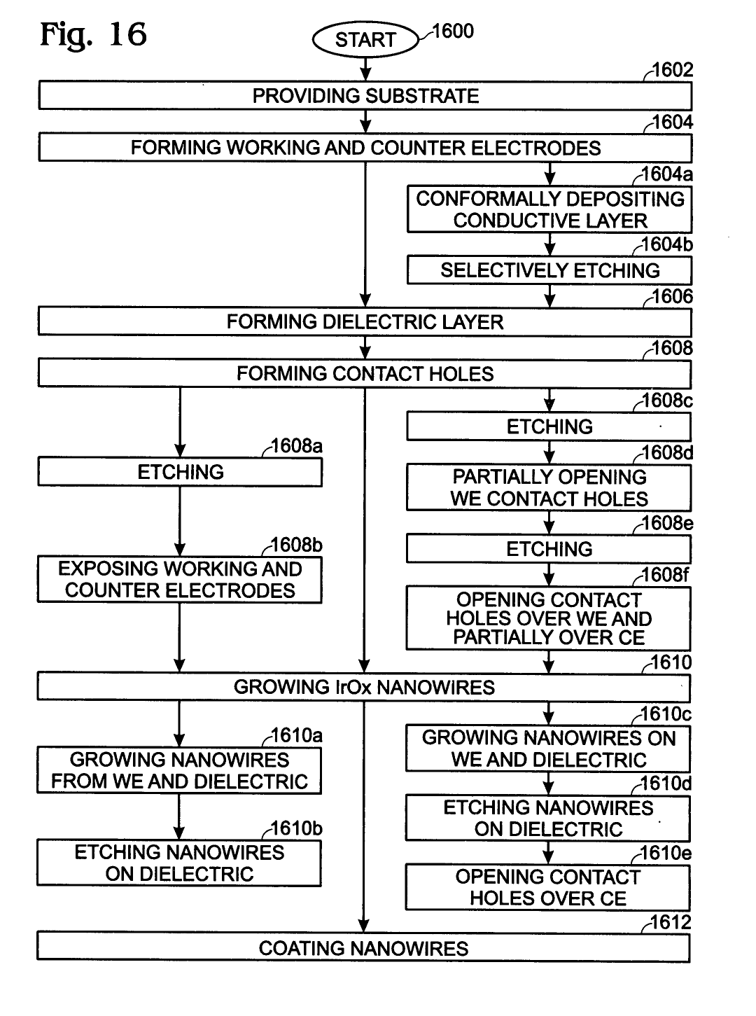

FIG. 16 is a flowchart illustrating a method for forming an iridium oxide (IrOx) nanowire protein sensor. Although the method is depicted as a sequence of numbered steps for clarity, the numbering does not necessarily dictate the order of the steps. It should be understood that some of these steps may be skipped, performed in parallel, or performed without the requirement of maintaining a strict order of sequence. The method starts at Step 1600.

Step 1602 provides a substrate from a material such as Si, SiO₂, quartz, glass, or polyimide. Step 1604 forms a working electrode and a counter electrode overlying the substrate, from a material such as ITO, SnO₂, ZnO, TiO₂, doped ITO, doped SnO₂, doped ZnO, doped TiO₂, TiN, TaN, Au, Pt, or Ir. Step 1606 forms a dielectric layer overlying the working and counter electrodes, from a material such as SiO₂ or SiN. Step 1608 forms contact holes in the dielectric layer, exposing regions of the working and counter electrodes. Typically, the contact holes have openings about equal to, and aligned with top surfaces of the underlying working and counter electrodes. Step 1610 grows IrOx (0<X≦2) nanowires from exposed regions of the working electrode. In one aspect, Step 1610 also grows IrOx nanowires from exposed regions of the counter electrode. Optionally, Step 1612 coats the IrOx nanowires with antibody linker molecules, antibodies, or protein blocker agents. In some aspects, the nanowires may be covered with combinations of the above-mentioned materials. For example, linkers may be used with blockers to reduce the nonspecific binding of proteins. In some aspects, an antibody coating may act as a binding for a specific protein.

In one aspect, forming contact holes in the dielectric layer includes substeps. Step 1608a selectively etches the dielectric layer, and Step 1608b exposes the working and counter electrodes. Then, growing IrOx nanowires from the exposed regions of the working electrode includes substeps. Step 1610a grows IrOx nanowires from the regions of the working electrode exposed by the contact hole, and also grows IrOx nanowires on the dielectric. Step 1610b etches the IrOx nanowires grown on the dielectric.

In a different aspect, Step 1608c selectively etches the dielectric layer. Step 1608d partially opens contact holes overlying the working electrode. Step 1608e selectively etches the dielectric layer. Step 1608f opens contact holes overlying the working electrode, and partially opens contact holes overlying the counter electrode. Then, Step 1610c grows IrOx nanowires from the exposed regions of the working electrode, and also grows IrOx nanowires on the dielectric. Unlike Step 1610a, dielectric still covers the counter electrode. Step 1610d etches the IrOx nanowires grown on the dielectric, and Step 1610e opens contact holes overlying the counter electrode.

In another aspect, providing the substrate in Step 1602 includes providing a substrate chip with edges. Then, forming the working electrode and counter electrode includes substeps. Step 1604a conformally deposits a conductive layer overlying the substrate. Prior to forming the dielectric layer, Step 1604b selectively etches the conductive layer to form working and counter electrodes, probe pads along the chip edges, and traces connecting the electrodes to the probe pads. The working electrode and the counter electrode made be formed in an orientation such as adjacent, the counter electrode substantially surrounding the working electrode, and in an interdigital separation pattern. Typically, the working electrode and the counter electrodes are separated by a distance in a range between 0.1 and 10 microns.

The counter electrode may have a top surface area in a range of 1 square micron to 1 square millimeter (mm²), with a shape such as a circle, rectangle, hexagon, or oval. The working electrode likewise may be shaped as a circle, rectangle, hexagon, or oval, with a top surface area in the range of about 1 to 1000 times smaller than the counter electrode top surface area.

In one aspect, providing the substrate chip in Step 1602 includes providing a chip having a surface area in the range of 1 mm² to 1000 mm². Forming the working and counter electrodes in Step 1604 includes forming an array of working/counter electrode pairs on the substrate, where the array includes between 2 and 128 electrode pairs. Each electrode pair is separated by a (minimum) distance in the range of 1 to 500 microns, and arranged in a pattern such as a circle, concentric rings, or a grid.

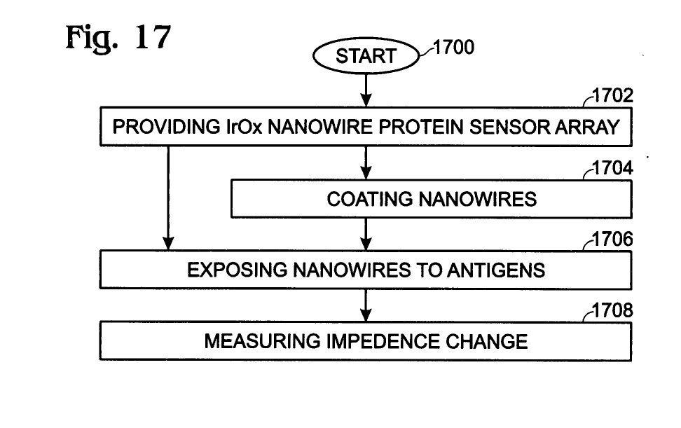

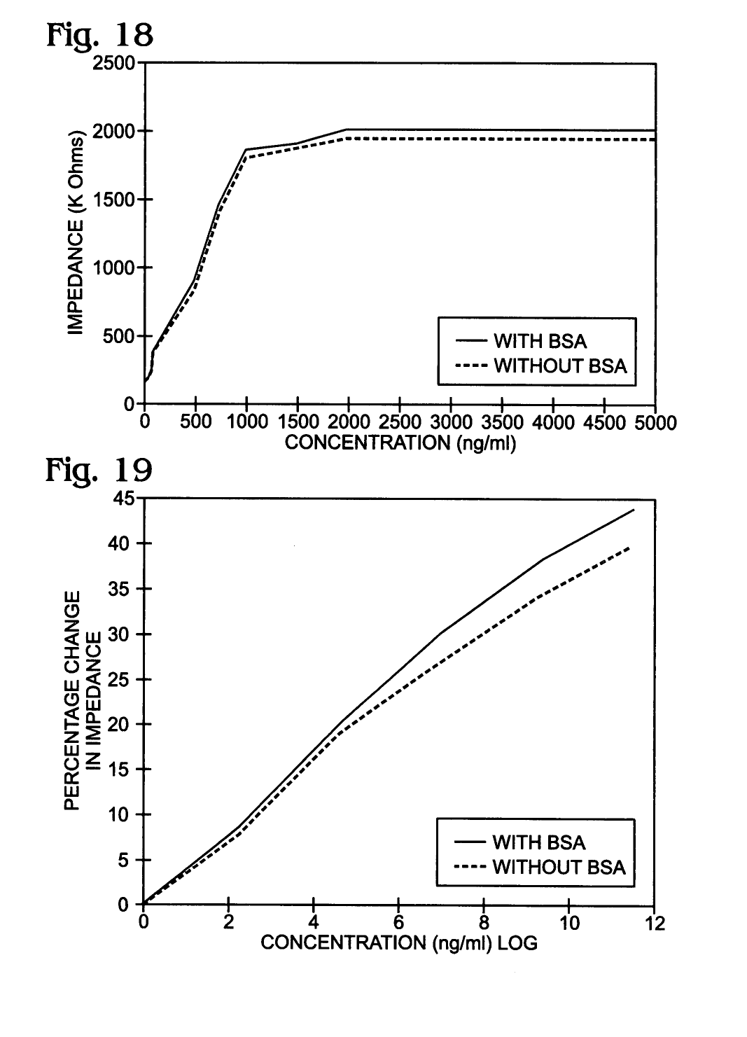

FIG. 17 is a flowchart illustrating a method for using capacitance measurements to detect the presence of proteins in an ambient environment. The method begins at Step 1700. Step 1702 provides a protein detector array on a substrate chip with a plurality of working/counter electrode pairs exposed by contact holes in a dielectric covering. As described above, IrOx (0<X≦2) nanowires are grown from regions of the working electrode exposed by contact holes. In some aspects, IrOx nanowires are also grown from regions of the counter electrode exposed by contact holes. Step 1704 coats the IrOx nanowires with antibody linker molecules, antibodies, protein blocking agents, or combinations of the above-mentioned materials. Note: Step 1704 is not performed if the nanowires are pre-coated. In another aspect, the method is performed without coating the nanowires. That is, Step 1704 may not be performed, even if the nanowires are not pre-coated.

Step 1706 exposes the IrOx nanowires (with or without coating) to an ambient environment including antigen molecules. In response to the antigen molecules binding to the nanowires, Step 1708 measures a change in impedance between the working and counter electrodes. As noted in more detail above, Step 1708 measures a change in impedance (e.g., a maximum impedance) at a first frequency. Alternately stated, the method measures local minimum or maximum electrical characteristics at particular frequencies, which are known to be associated with particular proteins.

In one aspect, coating the IrOx nanowires with antibody linker molecules in Step 1704 includes coating with a material such as alkanethiols, carboxylic acids, organosilicon derivatives, or diphosphonates. Alternately, if the coating is a blocker, it may be BSA. After coating, Step 1704 may include heating the substrate to a temperature in the range of 20° to 60° C., for a duration in the range of about 15 to 60 minutes.

Additional details of the IrOx nanowire fabrication process can be found in the following related pending applications:

OPTICAL DEVICE WITH IrOx NANOSTRUCTURE ELECTRODE NEURAL INTERFACE, invented by Zhang et al, Ser. No. 11/496,157, filed Jul. 31, 2006, Attorney Docket No. SLA8084;

Iridium Oxide Nanotubes and Method for FORMING SAME, invented by Zhang et al., Ser. No. 10/971,280, filed Oct. 21, 2004, Attorney Docket No. SLA0901; and,

Iridium Oxide Nanowire and Method for FORMING SAME, invented by Zhang et al., Ser. No. 10/971,330, filed Oct. 21, 2004, Attorney Docket No. SLA0903.

IrOx NANOWIRE NEURAL SENSOR, invented by Zhang et al., Ser. No. 11/809,959, filed Jun. 4, 2007, Attorney Docket No. SLA2145.

The four above-mentioned applications are incorporated herein by reference.

An IrOx nanowire protein sensor array and corresponding fabrication processes have been provided. Examples of specific materials, process steps, and structures have been presented to illustrate the invention. However, the invention is not limited to merely these examples. Other variations and embodiments of the invention will occur to those skilled in the art.

We claim:

1. A method for forming an iridium oxide (IrOx) nanowire protein sensor, the method comprising:
providing a substrate;
forming a working electrode and a counter electrode overlying the substrate;
forming a dielectric layer overlying the working and counter electrodes;
forming contact holes in the dielectric layer, exposing regions of the working and counter electrodes; and,
growing IrOx (0<X≦2) nanowires from exposed regions of the working electrode.

2. The method of claim 1 further comprising:
growing IrOx nanowires from exposed regions of the counter electrode.

3. The method of claim 1 wherein forming the working and counter electrode includes forming the electrodes from a material selected from a group consisting of ITO, SnO₂, ZnO, TiO₂, doped ITO, doped SnO₂, doped ZnO, doped TiO₂, TiN, TaN, Au, Pt, and Ir.

4. The method of claim 1 wherein providing the substrate includes providing a substrate material selected from a group consisting of Si, SiO₂, quartz, glass, and polyimide.

5. The method of claim 1 wherein providing the substrate includes providing a substrate chip with edges;
wherein forming the working electrode and counter electrode includes:
conformally depositing a conductive layer overlying the substrate; and,
prior to forming the dielectric layer, selectively etching the conductive layer to form working and counter electrodes, probe pads along the chip edges, and traces connecting the electrodes to the probe pads.

6. The method of claim 1 wherein forming contact holes in the dielectric layer includes:
selectively etching the dielectric layer;
exposing the working and counter electrodes;
wherein growing IrOx nanowires from the exposed regions of the working electrode includes:
growing IrOx nanowires from the regions of the working electrode exposed by the contact hole, and growing IrOx nanowires on the dielectric; and,
etching the IrOx nanowires grown on the dielectric.

7. The method of claim 1 wherein forming contact holes includes forming contact holes having openings about equal to, and aligned with top surfaces of the underlying working and counter electrodes.

8. The method of claim 1 wherein forming the working electrode and the counter electrode overlying the substrate includes forming a counter electrode having a top surface area in a range of 1 square micron to 1 square millimeter (mm²) and a shape selected from a group consisting of a circle, rectangle, hexagon, and oval, and a working electrode having a shape selected from a group consisting of a circle, rectangle, hexagonal, and oval, and a top surface area in a range of about 1 to 1000 times smaller than the counter electrode top surface area.

9. The method of claim 8 wherein forming the working electrode and the counter electrode overlying the substrate includes forming the electrodes in an orientation selected from a group consisting of adjacent electrodes, the counter electrode substantially surrounding the working electrode, and an interdigital separation pattern.

10. The method of claim 1 forming the working electrode and the counter electrode overlying the substrate includes forming working and counter electrodes separated by a distance in a range between 0.1 and 10 microns.

11. The method of claim 1 wherein forming contact holes in the dielectric layer includes:
selectively etching the dielectric layer;
partially opening contact holes overlying the working electrode;
selectively etching the dielectric layer;
opening contact holes overlying the working electrode and partially opening contact holes overlying the counter electrode;
wherein growing IrOx nanowires from the exposed regions of the working electrode includes:
growing IrOx nanowires from the exposed regions of the working electrode, and growing IrOx nanowires on the dielectric;
etching the IrOx nanowires grown on the dielectric; and,
opening contact holes overlying the counter electrode.

12. The method of claim 5 wherein providing the substrate chip includes providing a chip having a surface area in the range of 1 mm² to 1000 mm²; and,
wherein forming the working and counter electrodes includes forming an array of working/counter electrode pairs on the substrate, where the array includes between 2 and 128 electrode pairs, each pair separated by a distance in a range of 1 to 500 microns, and arranged in a pattern selected from a group consisting of a circle, concentric rings, and a grid.

13. The method of claim 1 wherein forming the dielectric layer includes forming a dielectric layer from a material selected from a group consisting of SiO₂ and SiN.

14. The method of claim 1 further comprising:
coating the IrOx nanowires with a material selected from a group consisting of antibody linker molecules, antibodies, protein blocker agents, and combinations of the above-mentioned materials.

15. The method for using capacitance measurements to detect the presence of proteins in an ambient environment, the method comprising:
providing a protein detector array on a substrate chip with a plurality of working/counter electrode pairs exposed by contact holes in a dielectric covering, with IrOx (0<X≦2) nanowires grown from regions of the working electrode exposed by contact holes;
exposing the IrOx nanowires to an ambient environment including antigen molecules; and,
in response to the antigen molecules binding to the IrOx nanowires, measuring a change in impedance between the working and counter electrodes.

16. The method of claim 15 wherein providing the protein sensor array includes providing an array with IrOx nanowires grown from regions of the counter electrodes exposed by contact holes.

17. The method of claim 15 further comprising:
coating the IrOx nanowires with a material selected from a group consisting of antibody linker molecules, antibodies, protein blocker agents, and combinations of the above-mentioned materials.

18. The method of claim 17 wherein coating the IrOx nanowires with antibody linker molecules includes coating with a material selected from a group consisting of alkanethiols, carboxylic acids, organosilicon derivatives, and diphosphonates; and,
wherein coating the IrOx nanowires with a protein blocker agent includes coating with bovine serum albumin (BSA).

19. The method of claim 15 wherein coating the IrOx nanowires with antibody linker molecules includes heating the substrate to a temperature in a range of 20° to 60° C., for a duration in a range of about 15 to 60 minutes.

20. The method of claim 15 wherein measuring the change in impedance between the working and counter electrodes includes measuring a change in impedance at a first frequency.

21. An iridium oxide (IrOx) nanowire protein sensor array, the sensor array comprising:
a substrate;
a plurality of electrode pairs, each electrode pair including:
a working electrode overlying the substrate;
a counter electrode overlying the substrate;
a dielectric layer overlying the working and counter electrodes;
contact holes in the dielectric layer, exposing regions of the working and counter electrodes; and,
IrOx (0<X≦2) nanowires grown from exposed regions of the working electrode.

22. The sensor array of claim 21 wherein each electrode pair further includes:
IrOx nanowires grown from exposed regions of the counter electrode.

23. The sensor array of claim 21 wherein the working and counter electrodes are a material selected from a group consisting of ITO, SnO₂, ZnO, TiO₂, doped ITO, doped SnO₂, doped ZnO, doped TiO₂, TiN, TaN, Au, Pt, and Ir.

24. The sensor array of claim 21 wherein the substrate is a material selected from a group consisting of Si, SiO₂, quartz, glass, and polyimide.

25. The sensor array of claim 21 wherein the substrate is a substrate chip with edges; and,
wherein each electrode pair further includes probe pads along the chip edges, and traces connecting the electrodes to the probe pads.

26. The sensor array of claim 21 wherein the contact holes have openings about equal to, and aligned with top surfaces of the underlying working and counter electrodes.

27. The sensor array of claim 21 wherein each counter electrode has a top surface area in a range of 1 square micron to 1 square millimeter (mm²) and a shape selected from a group consisting of a circle, rectangle, hexagonal, and oval; and
wherein each working electrode has a shape selected from a group consisting of a circle, rectangle, hexagon, and oval, and a top surface area in a range of about 1 to 1000 times smaller than the counter electrode top surface area.

28. The sensor array of claim 27 wherein the working electrode and the counter electrode are arranged in an orientation selected from a group consisting of adjacent, the counter electrode substantially surrounding the working electrode, and an interdigital separation pattern.

29. The sensor array of claim 21 wherein each working electrode is separated from its corresponding counter electrode by a distance in a range between 0.1 and 10 microns.

30. The sensor array of claim 25 wherein the substrate chip has a surface area in the range of 1 mm² to 1000 mm²; and,
wherein the array includes between 2 and 128 electrode pairs, each pair separated by a distance in a range of 1 to 500 microns, and arranged in a pattern selected from a group consisting of a circle, concentric rings, and a grid.

31. The sensor array of claim 21 wherein the dielectric layer is a material selected from a group consisting of SiO₂ and SiN.

32. The sensor array of claim 21 wherein each electrode pair further includes a coating of a material selected from a group consisting of antibody linker molecules, antibodies, protein blocker agents, and combinations of the above-mentioned materials.
